(19) United States
(12) Patent Application Publication
(10) Pub. No.: US 2010/0322947 A1
(43) Pub. Date: Dec. 23, 2010

(54) PHARMACEUTICAL COMPOSITION FOR TREATING AVELLINO CORNEA DYSTROPHY COMPRISING AN ANTIBODY AGAINST TGF-BETA

(76) Inventors: Eung Kweon Kim, Seoul (KR); Nae-Choon Yoo, Seoul (KR); Won-Min Yoo, Seoul (KR)

Correspondence Address: INTELLECTUAL PROPERTY / TECHNOLOGY LAW, PO BOX 14329, RESEARCH TRIANGLE PARK, NC, 27709, US

(21) Appl. No.: 12/858,241

(22) Filed: Aug. 17, 2010

Related U.S. Application Data
(63) Continuation of application No. 11/817,023, filed on Apr. 7, 2008.

(30) Foreign Application Priority Data
Mar. 8, 2005 (KR) 10-2005-0018942

(51) Int. Cl. A61K 39/395; A61P 27/02

(52) U.S. Cl. 424/158.1

(57) ABSTRACT

The present invention relates to a medicine for treating Avellino corneal dystrophy (ACD), and more particularly, to a pharmaceutical composition for treating Avellino corneal dystrophy containing an antibody against TGF-β as an effective ingredient. The pharmaceutical composition of the present invention has an effect of improving symptoms of a patient with severe Avellino corneal dystrophy due to TGF-β induced by exposure to intense light, such as UV etc.

### CROSS REFERENCE TO RELATED APPLICATIONS

This application is a continuation under 35 U.S.C. §120 of U.S. patent application Ser. No. 11/817,023 filed Aug. 24, 2007, which is a U.S. national phase under 35 U.S.C. §371 of International Patent Application No. PCT/KR06/00819 filed Mar. 8, 2006, which in turn claims priority under 35 U.S.C. §119 of Korean Patent Application No. 10-2005-0018942 filed Mar. 8, 2005. The disclosures of all of the foregoing applications are hereby incorporated herein by reference in their respective entireties, for all purposes.

### TECHNICAL FIELD

The present invention relates to a medicine for treating Avellino corneal dystrophy (ACD), and more particularly, to a pharmaceutical composition for treating Avellino corneal dystrophy comprising an antibody against TGF-β as an effective ingredient.

### BACKGROUND ART

Avellino corneal dystrophy is a hereditary disease which white granules, hyaline in the cornea of the eye forms milky deposits, so that the cornea becomes blurry to cause bad visual acuity, and thus leading to the loss of eyesight. This disease is generated by point mutation in which codon CGC (arginine) corresponding to 124^{th} amino acid in βIG-H3 gene is replaced by CAC (histidine) (Munier, F. L. et al., Nat. Genet., 15:247, 1997). All people with this abnormal gene show symptom and the symptom starts to show from the juvenile period. Recently, Avellino corneal dystrophy has been recognized since LASIK surgery becomes more popular and the cornea is damaged by UV radiation and thus the disease rapidly progresses to Avellino corneal dystrophy.

After Avellino corneal dystrophy is first known in 1998 (Holland, E. J. et al., Opthalmology, 99:1564, 1992; Kennedy, S. M. et al., Br. J. Opthalmol., 80:489, 1996; Dolmetsch, A. M. et al., Can. J. Opthalmol. 31:29, 1996; Afashari, N. A. et al., Arch. Opthalmol., 119:16, 2001; Stewart, H. S., Hum. Mutat., 14:126, 1999), biochemical researches on βIG-H3 protein are recently being reported (Kim, J. E. et al., Investigative Opthalmology & Visual Science, 43:3, 2002; Park, S. J. et al., Peptides, 25:199, 2004). But until now, there has been no development of significant therapeutic agents. According to the present inventor's research, if a patient who has had LASIK surgery is identified as a heterozygote for the Avellino corneal dystrophy gene, Avellino corneal dystrophy develops or progresses rapidly (Kim, E. K. et al., Cornea, 21:223, 2002; Kim, E. K. et al., Opthalmology, 111:463, 2004).

Therefore, there is an urgent need for the development of a medicine and/or a therapeutic method which can treat Avellino corneal dystrophy, but there has not been any report, yet.

Accordingly, the present inventors have made extensive efforts to develop a medicine for treating Avellino corneal dystrophy, as a result, we found that the expression of βIG-H3 protein is increased when a corneal stromal cell is treated with TGF-β and Avellino corneal dystrophy symptom gets aggravated by the increase of TGF-β expression, at the same time, confirmed that the expression of increased βIG-H3 protein can become normal when the corneal stromal cell is treated with an antibody against TGF-β, thereby completing the present invention.

### SUMMARY OF INVENTION

The main object of the present invention is to provide a pharmaceutical composition for treating Avellino corneal dystrophy, which administers to the cornea of a patient with Avellino corneal dystrophy to be able to mitigate symptom.

To achieve the above object, the present invention provides a pharmaceutical composition for treating Avellino corneal dystrophy containing an antibody against TGF-β as an effective ingredient.

Other features and embodiments of the present invention will be more fully apparent from the following detailed description and appended claims.

### BRIEF DESCRIPTION OF DRAWINGS

FIG. 1 shows an electron microscope photograph of a corneal stromal cell located on corneal flap surface through which a microkeratome has passed.

FIG. 2 shows a graph showing an increased amount of TGF-β when a human fibroblast is irradiated by UV-B.

FIG. 3 shows a photograph showing staining for βIG-H3 protein formed in a corneal stromal cell of a homozygote.

FIG. 4 shows a change in βIG-H3 protein expression quantity in a normal corneal stromal cell and a corneal stromal cell of an Avellino corneal dystrophy homozygote by western blot (left column: a normal corneal stromal cell; right column: a corneal stromal cell of an Avellino corneal dystrophy homozygote; lane 1: control; lane 2: a group treated with TGF-β; lane 3: a group treated with TGF-(and an antibody against TGF-β.

### DETAILED DESCRIPTION OF THE INVENTION AND EMBODIMENTS

In the present invention, from the fact that Avellino corneal dystrophy is aggravated by light stimulation (for example, ultraviolet rays, etc.), it is confirmed that the amount of TGF-β in cells was increased by UV irradiation, and the expression of the mutant protein, βIG-H3 protein which causes Avellino corneal dystrophy was increased by TGF-β to cause the accumulation of βIG-H3 protein in cells. Also, it was confirmed that the expression of βIG-H3 protein which has been increased by TGF-β was decreased if a corneal stromal cell was treated with an antibody against TGF-β.

As a result, it was confirmed that the antibody against TGF-β was useful for treating Avellino corneal dystrophy. Therefore, the present invention provides a pharmaceutical composition for treating Avellino corneal dystrophy containing the antibody against TGF-β as an effective ingredient.

Additionally, the present invention provides a pharmaceutical composition containing the antibody against TGF-β pharmacologically as an effective ingredient, together with an opthalmologically approved carrier.

In the pharmaceutical composition of the present invention, the content of the antibody against TGF-β isn't limited as far as it is useful for treating, but it is preferable to generally contain 0.0000001-10% by weight per total composition.

The pharmaceutical composition of the present invention can contain components of adjuvants etc. including a buffer, an antimicrobial preserving agent, a surfactant, an additional pharmaceutical, an antioxidant, a tonic regulator, an antiseptic, a thickener and a viscosity improver.

In the present invention, any buffer among proper buffers, which harmonize with other substances of liquid preparations in the field of opthalmology and doesn't show harmful characteristic or toxicity that can damage eyes, can be used as the buffer. The proper buffers include boric acid, sodium boric acid, sodium phosphate (including 1, 2 and 3 basic phosphate, such as 1 basic sodium phosphate 1 hydrate, 2 basic sodium phosphate 7 hydrate and mixtures thereof). Any other proper buffers can be used to stabilized pH level of the ophthalmic liquid medicine by conferring physiological pH approved for ophthalmic liquid medicines. Since said buffers are just examples and these buffers are well known in opthalmologic field, a person skilled in the art can choose proper buffers that can be used for the composition of the present invention.

In the present invention, the preferable examples of the antimicrobial preserving agent include benzalchonium chloride, timerosal, chlorobutanol, methyl paraben, prophyl paraben, phenylethyl alchol, edetate disodium, sorbic acid, ONAMER M. It is preferable to use the above preserving agents at about 0.001-1% by weight per total composition.

The pharmaceutical composition of the present invention can be prepared in various formulations, such as liquid, suspension, emulsion, gel and a solid form of insert into eyes as a person skilled in the art can easily recognizes.

pH of the pharmaceutical composition of the present invention is preferable to be pH 6.8-8.0 which corresponds to pH of eye liquid or at which eyes have resistance without uncomfortableness or inflammation, and more preferably about pH 7.0-7.8. To stabilize an ophthalmic liquid medicine at a desirable pH level, small amount of effective buffer is mixed. An effective amount of buffer administered to buffer an ophthalmic liquid medicine at about pH 6.8-8.0 can be broadly varied and determined according to a specific buffer used and a chemical composition of the pharmaceutical composition. But, to stabilize this liquid medicine at approved physiological pH, preferable result can be obtained when the amount of buffer mixed in the ophthalmic liquid medicine is about 0.05-1% weight/volume.

The osmotic pressure of the pharmaceutical composition of the present invention is preferable generally about 1-400 mOsM, and more preferably 260-340 mOsM. If necessary, the osmotic pressure can be adjusted using salt or drug vehicle approved in physiology and opthalmology. NaCl is suitable to approach physiological saline solution. The amount of NaCl added is preferably 0.01-1% by weight based on the total weight of the composition, and it is more preferable to be added in a range of 0.05%-0.45% by weight. To achieve the osmotic concentration of the above range, an equivalent amount of at least one salt comprised of anions, such as potassium, ammonium and cations, such as chloride, citrate, ascorbate, borate, phosphate, bicarbornate, sulfate, tiosulfate, bisulfate, sodium bisulfate, ammonium sulfate can be used together with NaCl or in place of NaCl. Also, sugar, such as mannitol, denstrose, sorbitol, glucose can be used to adjust the osmotic concentration.

### EXAMPLES

The present invention will hereinafter be described in further detail by examples. It will however be obvious to a person skilled in the art that these examples are given for illustrative purpose only, and the present invention is not limited to or by the examples.

### Example 1

### Observation of the Corneal Flap of the Avellino Cornea Dystrophy Patients

When a lesion in the corneal flap of an Avellino corneal dystrophy patient obtained after LASIK surgery was observed by scanning confocal electron microscopy, it was found that a stromal cell has gotten abnormally hypertrophic and abnormal proteins were not secreted to remain in cells and stagnate (FIG. 1). FIG. 1 shows a photograph of electron microscopy of a corneal stromal cell located on corneal flap surface through which a microkeratome has passed. A cyst (left) which is similar size to the cell (right) in photograph A was observed and it seems that abnormal proteins are accumulated in a cell to form the cyst. FIG. 1B shows a photograph of the electron microscopy of the moment when the cyst is ruptured, in which a cell membrane and abnormal proteins being exposed through ruptured cell membrane were observed.

From the above result, it can be confirmed that the cause of Avellino corneal dystrophy is trafficing which is caused since proteins are not normally secreted outside cells. Trafficing is known as a mechanism causing Alzheimer's disease etc. and it has not yet been studied in detail.

Also, as a result of observing the lesions of more than 500 Avellino corneal dystrophy patients, it was confirmed that all Avellino corneal dystrophy symptoms started from the corneal region exposed even when they squint their eyes in response to light stimulus. It means that Avellino corneal dystrophy is aggravated by external stimulus.

### Example 2

### Change of TGF-β Expression by UV-B Stimulus

In this example, whether the expression of TGF-β is increased by irradiating UV-B which is the most toxic to cells among components of light was examined. That is, a hTERT inactivated human corneal stromal cell was irradiated with UV-B light at the intensity of 10, 15 and 30 mJ/cm² UVB (Jaster, J V et al., Invest. Opthalmol. Vis. Sci., 44:1850, 2003) to measure the amount of TGF-β1 protein expression with time.

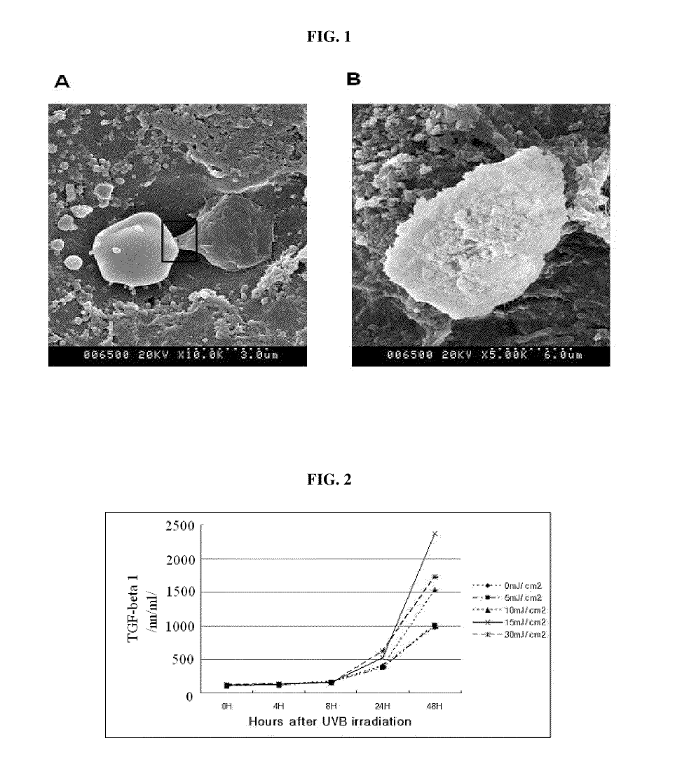

As a result, as shown in FIG. 2, it was confirmed that the expression of TGF-β is induced by UV-B irradiation in the corneal stromal cell. In FIG. 2, y axis shows the concentration of TGF-β1 in culture supernatant of a hTERT inactivated human corneal stromal cell after UV-B irradiation. As a result of examining by ELISA, when 24 and 48 hours have passed after 10, 15 and 30 mJ/cm² UV-B irradiation (ANCOVA: p<0.05), it was suggested that the expression of TGF-β protein was increased as compared with a control.

### Example 3

### Establishment of Corneal Stromal Cell Line

To confirm the effect of TGF-β increased by UV stimulus on the expression of βIG-H3 protein of a corneal stromal cell, corneal stromal cells were isolated in the corneal flaps of a homozygote of an Avellino corneal dystrophy patient and a normal patient to culture primarily.

After corneal endothelium was removed from the corneal flap obtained from a patient after LASIK surgery using forceps and the corneal flap, from which the endothelium was removed, was repetitively washed in serum free medium (K-SFM; Invitrogen-Gibco, USA) for corneal stromal cells containing supplement (25 mg of bovine pituitary extract and 2.5 μg of hrEGF; Invitrogen-Gibco, USA), the corneal flap was allowed to react in 0.5 mL of dispase (25 U/mL; Roche, USA) at 4 overnight.

After reaction, the endothelium was removed using forceps and stromal layer was cut to pieces to be subject to a reaction in solution containing 3 ml of K-SFM and 1000 U/ML of collagenase type for 20 minutes −2 hours. Tissues after the reaction were dispersed in 75 cm² of tissue culture flask containing 3 mL of DMEM (Invitrogen-Gibco, USA) supplemented with 20% fetal bovine serum 1× antibiotic-antimycotic (Invitrogen-Gibco, USA), 10 mM HEPES, 1.5% sodium bicarbonate, 0.004 N sodium hydroxide and 0.2 μg/mL kanamycin (Sigma-Aldrich, USA). After cells were grown to 100% of the flask capacity, the cells were dispersed in 25 cm² flask containing DMEM supplemented with 10% fetal bovine serum and cultured until the flask was filled with the cells at about 80% of the flask capacity to obtain the corneal stromal cell line of an Avellino corneal dystrophy homozygote and a normal corneal stromal cell line.

### Example 4

### Distribution of βIG-H3 Protein in Corneal Stromal Cell

To examine the Distribution of βIG-H3 protein in the corneal stromal cell of an Avellino corneal dystrophy homozygote cultured in Example 3, βIG-H3 protein was stained using βIG-H3 protein antibody labelled with FITC.

As a result, as shown in FIG. 3, it was confirmed that distribution of βIG-H3 protein was similar to that of secretory vesicle, and FIG. 3B showed that a part of cells in FIG. 3A was damaged to expose with βIG-H3 protein tangled and βIG-H3 protein was also observed in cells which have not been damaged.

From this result, it can be confirmed that Trafficing of βIG-H3 protein in cells can be caused in secretory vesicle.

### Example 5

### Change of βIG-H3 Protein Expression in Corneal Stromal Cell According to Treatment with TGF-β and an Antibody Against TGF-β

To examine βIG-H3 protein expression in a corneal stromal cell by treating with TGF-β and an antibody against TGF-β, a normal corneal stromal cell and a corneal stromal cell of an Avellino corneal dystrophy homozygote established in Example 3 were preincubated in DMEM without serum for 24 hours.

After each stromal cell was divided into a control, a group treated with TGF-β and a group treated with (TGF-β+ an antibody against TGF-β) a group treated with TGF-β and a group treated with (TGF-β+ an antibody against TGF-β) were treated with TGF-β (RND, USA) at a concentration of 10 ng/mL, and an antibody against TGF-β (human recombinant monoclonal TGF-(antibody, RND, USA) was treated at a concentration of 10 μg/mL. All cells treated were allowed to react in a medium without serum for 24 hours.

After completing the reaction, the corneal cells were collected to electrophorese in a 10% SDS-PAGE gel, followed by transferring to PVDF membrane and performing western blot using βIG-H3 protein antibody.

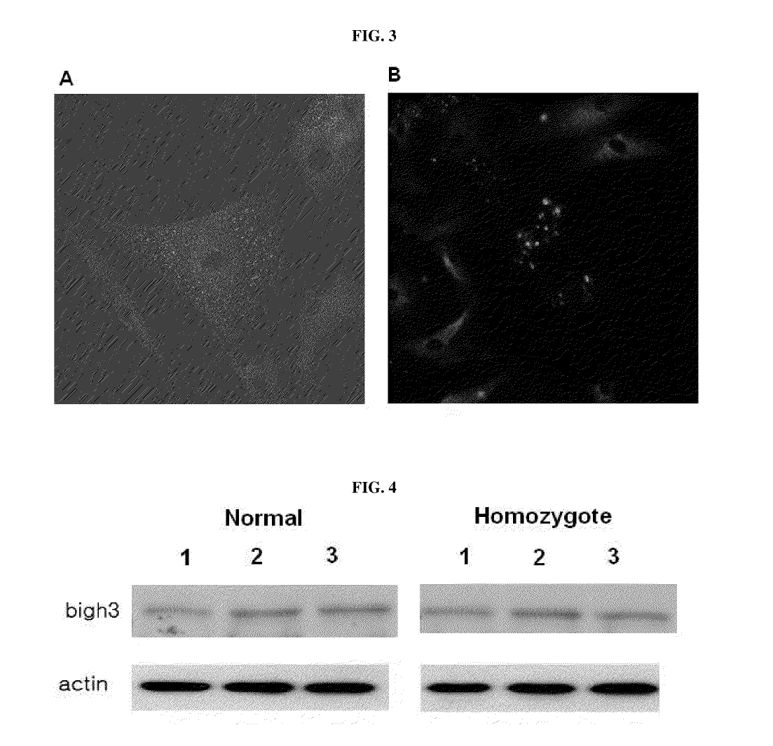

As a result, as shown in FIG. 4, it was confirmed that when corneal stromal cells were treated with TGF-β, the expressions of βIG-H3 protein were increased in a normal corneal stromal cell and a corneal stromal cell of an Avellino corneal dystrophy homozygote. Also, when corneal stromal cells were treated together with (TGF-β+ an antibody against TGF-β) it was confirmed that βIG-H3 protein expression which has been increased by TGF-β treatment was decreased.

From this result, it was suggested that an antibody against TGF-β suppressed βIG-H3 protein expression which is increased by TGF-β. Therefore, it was confirmed that symptom aggravation by the stimulus could be prevented with the administration of an antibody against TGF-β even if TGF-β increases by external stimulus of UV etc. in the corneal stromal cells of an Avellino corneal dystrophy patient.

Although the present invention has been described in detail with reference to the specific features, it will be apparent to those skilled in the art that this description is solely for a preferred embodiment and does not limit the scope of the present invention. Thus, the substantial scope of the present invention will be defined by the appended claims and equivalents thereof.

### INDUSTRIAL APPLICABILITY

As described above in detail, the present invention has an effect of providing a pharmaceutical composition for treating Avellino corneal dystrophy, which is administered to the cornea of the patient with Avellino corneal dystrophy to be able to mitigate symptom. The pharmaceutical composition of the present invention has an effect of improving symptoms of the patient with severe Avellino corneal dystrophy due to TGF-β induced by exposure to intense light, such as UV etc.

What is claimed is:

1. A method for treating Avellino corneal dystrophy in a subject, comprising the step of administering an effective amount of an antibody against TGF-β to the subject.

2. The method according to claim 1, wherein the step of administering an effective amount of an antibody against TGF-β further comprises administration of one or more adjuvants selected from the group consisting of buffers, anti-microbial preserving agents, surfactants, antioxidants, tonic regulators, antiseptics, thickeners and viscosity improvers.

3. A method for inhibiting expression of βIG-H3 in a subject, comprising the step of administering an antibody against TGF-β to the subject.

4. The method according to claim 3, wherein the step of administering an antibody against TGF-β is administered with one or more adjuvants selected from the group consisting of buffers, anti-microbial preserving agents, surfactants, antioxidants, tonic regulators, antiseptics, thickeners and viscosity improvers.
